(19) United States
(12) Patent Application Publication
(10) Pub. No.: US 2019/0262598 A1
(43) Pub. Date: Aug. 29, 2019

(54) HEMOSTASIS VALVE ALLOWING FOR LATERAL TRANSLATION AND SIMULTANEOUS ASPIRATION

(71) Applicants: STRYKER CORPORATION, Kalamazoo, MI (US); STRYKER EUROPEAN HOLDINGS I, LLC, Kalamazoo, MI (US)

(72) Inventors: Thomas J. Mock, Emeryville, CA (US); Alice Bradbury Welsh Brochu, Pleasanton, CA (US)

(21) Appl. No.: 16/274,989

(22) Filed: Feb. 13, 2019

Related U.S. Application Data
(60) Provisional application No. 62/636,590, filed on Feb. 28, 2018.

(51) Int. Cl. A61M 39/06

(52) U.S. Cl. CPC A61M 39/06; A61M 25/0662

(57) ABSTRACT

A valve apparatus having a combination of a slit seal and a compressive seal to allow for lateral translation of a catheter running through the valve apparatus without compromising on a vacuum created in the valve apparatus, but also allowing for the catheter or guidewire to be locked in place, as needed. The slit seal allows lateral translation while maintaining a vacuum, and the compressive seal allows for locking of the catheter or guidewire in place.

### RELATED APPLICATION DATA

The present application claims the benefit under 35 U.S.C. § 119 to U.S. provisional patent application Ser. No. 62/636,590, filed Feb. 28, 2018. The foregoing application is hereby incorporated by reference into the present application in its entirety.

### FIELD

The invention disclosed herein relate generally to medical devices and, more particularly, to hemostasis valves intended for use with catheters to be inserted in a patient's vasculature system.

### BACKGROUND

Many surgical procedures require use of catheters and/or guidewires introduced into, and navigated within, the cardiovascular system of a patient. Common catheter designs include an elongated and cylindrical catheter body having a passageway or lumen therethrough for fluid flow and/or introduction of an implantable device. For example, in one type of use, an end of the catheter is inserted into the body of the patient through an incision in a blood vessel. The catheter may be advanced through the vessel until a distal end of the catheter is localized at a desired location in the body. A guidewire is a long, cylindrical wire that may be used for directing the catheter to the desired location within the body. It is typically smaller in diameter and more rigid as compared to the catheter. Using the guidewire as a guide, the catheter may be advanced along the length of the guidewire in order to position the catheter at the desired location.

Surgical procedures may involve the insertion and removal of several different types of catheters and/or guidewires. A common problem encountered when inserting and removing catheter is to control bleeding at the point in the body where these catheters or guidewires are introduced. To this end, to ensure easy insertion and removal of the catheter and/or guidewire, a distal end of an introducer is first secured within a large vessel (e.g., a femoral artery) of a patient. Such introducer is typically a large hollow tube that acts as a port to the cardiovascular system of the patient. The proximal end of the introducer is positioned outside the body and is attached to an adaptor, which typically comprises a short, rigid tube having a connector at one end to connect the passageway of the adaptor to the exposed end of the introducer, thereby allowing for fluids or other medical instruments to pass through the adaptor and into the introducer.

A hemostasis valve is commonly secured to the other end of the adaptor. Hemostasis valves are routinely used in many surgical procedures to minimize fluid loss during interventional and diagnostic procedures. The hemostasis valve may include an enlarged chamber position that is aligned to or connected to the passageway of the adaptor, wherein the chamber may include one or more seals that prevent the patient's blood from escaping out of the adaptor through the access of the valve. Hemostasis valves are offered in a variety of shapes and sizes to accommodate many needs and the seals are intended to work with various sizes of guidewires and catheters. Hemostasis valves typically connect to the back end of a catheter or sheath to allow for easy translation or introduction of other devices while minimizing blood loss and/or preventing air introduction into the catheter. In addition, hemostasis valves typically have a port that allows for flushing of saline and/or for aspiration. Ideally, hemostasis valves provide low resistance while advancing catheters or guidewires, but enough friction to secure the catheter or guidewire in place to prevent accidental movement.

Depending on the type of procedure, such as thrombectomy for acute ischemic stroke, it is sometimes important to laterally translate the catheter through the hemostasis valve, while maintaining aspiration or flow of fluid through the port. Removing catheters and guidewires through current hemostasis valves, especially those that compress down to “lock” the catheter in place, often result in a large loss of blood. In addition, they allow for air to enter into the system, potentially leading to air embolisms, and results in poor aspiration performance.

### SUMMARY

In accordance with an exemplary embodiment of the disclosed inventions, a valve apparatus, comprises a valve body having multiple detachable parts, respective housings of the multiple detachable parts configured to be connected together in an axial fashion to form the valve body, the valve body defining a lumen for passage of a catheter or a guidewire, the valve body having a proximal end and a distal end, the distal end configured to be adapted to a port connected to a cardiovascular or other intravenous system of a patient, a compressible seal having a lumen for the passage of the catheter of the guidewire, the compressible seal disposed within a screw thread portion of a first detachable part of the valve body, the first detachable part of the valve body connected to a second detachable part of the valve body in an axial, screw threaded manner, whereby, when the catheter or guidewire is present in the lumen, rotation of the first and second detachable parts relative to each other, causes them to advance and retract relative to each other, the compressible seal disposed to compress inside the first detachable part when the second detachable part is screwed into the first detachable part, thereby locking the catheter or guidewire in place, and a slit seal disposed between a third detachable part and the first detachable part, the slit seal disposed such that the catheter or the guidewire passes through the slit seal, maintaining a seal even when the catheter or guidewire undergoes lateral translation through the lumen of the valve body.

The slit seal may be (without limitation) cross-slit. The outer diameter of the compressible seal may be substantially equal to an inner diameter of a cavity formed at a proximal end of the inner screw body. In one or more embodiments, a diameter of the slit seal is substantially equal to an inner diameter of a cavity formed at a proximal end of the third detachable part. An inner diameter of the compressible seal is substantially similar to an outer diameter of the catheter or guidewire. The compressible seal may be made from a rubber material. The slit seal may be made from a silicone material. The compressible seal may be proximal in relation to the slit seal. The valve apparatus may be a hemostasis valve.

In accordance with another exemplary embodiment of the disclosed inventions, a valve apparatus having multiple detachable parts, wherein respective housings of the multiple detachable parts are configured to be connected together in an axial fashion to form a valve body, comprises a continuous lumen for passage of a catheter or guidewire through the valve body, a compressible seal positioned in between a first detachable part and second detachable part of the multiple detachable parts, the compressible seal having a first lumen continuous with the continuous lumen of the valve apparatus, whereby, when the catheter or guidewire is present in the lumen, a movement of the first detachable part and the second detachable part relative to one another, causes a radial compression force to be exerted on the compressive seal, thereby locking the catheter or guidewire in place, and a slit seal having a second lumen continuous with the continuous lumen of the valve apparatus, the slit seal maintaining a seal even when the catheter or guidewire undergoes lateral translation through the lumen of the valve body. Without limitation, the first detachable part and the second detachable part may comprise complementary screw thread grooves, and the movement may comprise screwing the first detachable part in relation to the second detachable part.

These and other aspects and embodiments of the disclosed inventions are described in more detail below, in conjunction with the accompanying figures.

### BRIEF DESCRIPTION OF THE FIGURES

The drawings illustrate the design and utility of embodiments of the disclosed inventions, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments of the disclosed inventions and are not therefore to be considered limiting of their scope.

FIG. 1 illustrates the valve apparatus having a combination of seals in accordance with exemplary embodiments of the disclosed inventions;

FIG. 2 illustrates a disassembled plain view and a disassembled perspective view of the valve apparatus in accordance with exemplary embodiments of the disclosed inventions; and

FIG. 3 illustrates one embodiment of the valve apparatus having a connecting tube that is orthogonal to a tubular body of the valve apparatus.

### DETAILED DESCRIPTION OF THE ILLUSTRATED EMBODIMENTS

All numeric values are herein assumed to be modified by the terms “about” or “approximately,” whether or not explicitly indicated, wherein the terms “about” and “approximately” generally refer to a range of numbers that one of skill in the art would consider equivalent to the recited value (i.e., having the same function or result). In some instances, the terms “about” and “approximately” may include numbers that are rounded to the nearest significant figure. The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. In describing the depicted embodiments of the disclosed inventions illustrated in the accompanying figures, specific terminology is employed for the sake of clarity and ease of description. However, the disclosure of this patent specification is not intended to be limited to the specific terminology so selected, and it is to be understood that each specific element includes all technical equivalents that operate in a similar manner. It is to be further understood that the various elements and/or features of different illustrative embodiments may be combined with each other and/or substituted for each other wherever possible within the scope of this disclosure and the appended claims.

Various embodiments of the disclosed inventions are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the disclosed inventions, which is defined only by the appended claims and their equivalents. In addition, an illustrated embodiment of the disclosed inventions needs not have all the aspects or advantages shown. For example, an aspect or an advantage described in conjunction with a particular embodiment of the disclosed inventions is not necessarily limited to that embodiment and can be practiced in any other embodiments even if not so illustrated.

A hemostasis valve having a plurality of sealing mechanisms will now be described. Advantageously, the hemostasis valve described herein comprises both a compressible seal and a slit seal, the combination of which allows for a catheter going through the hemostasis valve to be locked in place or laterally translated while simultaneously maintaining aspiration and/or preventing fluid loss. The compressible seal prevents the flow of fluids out of the hemostasis valve and locks the catheter or guidewire in place when compressive force is exerted on the compressible seal. Similarly, the slit seal or slit valve substantially prevents the flow of fluids or air out of the hemostasis valve, but does not lock the catheter in place. Thus, in combination, when the compressible seal is unlocked, the catheter can be laterally translated while substantially maintaining vacuum or preventing fluid loss, but as needed, the catheter can be locked in place by exerting radial force on the compressible seal.

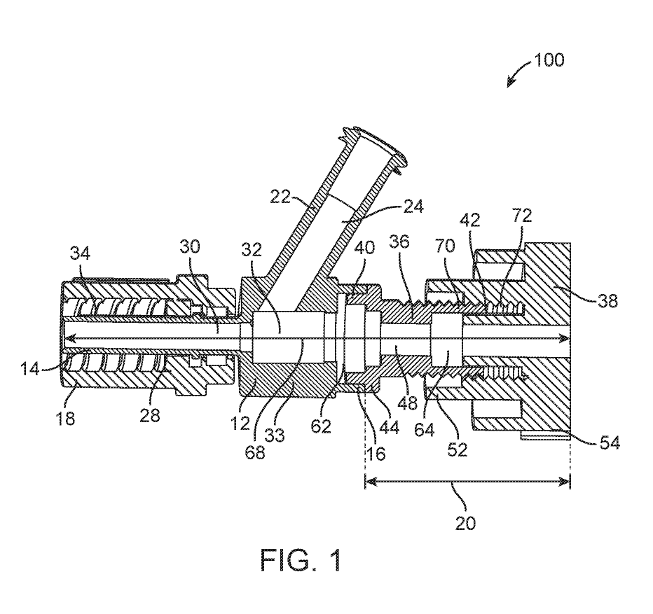

Referring now to the cross-sectional view of the hemostasis valve illustrated in FIG. 1, the hemostasis valve 100 comprises a tubular body 12, a rotatable connector 18, and a valve assembly 20. In one or more embodiments, the tubular body 12 comprises a distal end 14 and an opposing proximal end 16. On the distal end 14, the tubular body defines a tube 28, an outer diameter of which is smaller than the outer diameter of the tubular body 12 at the proximal end 16. The tube 28 defines a lumen 30 that extends into a slightly larger lumen 32 in a middle section 33 of the tubular body 12. The tube 28 is configured to fit into a lumen 34 defined by the rotatable connector 18.

The tubular body 12 may include a connector tube 22. In some embodiments, the connector tube 22 may have a central lumen 24 formed within such that it is in fluid communication with the lumen 32 of the tubular body 12. As will be seen further below, the connector tube 22 may be orthogonal to the tubular body in some embodiments. In other embodiments, the connector tube 22 may be disposed at a desired angle away from the tubular body 12, as shown in the illustrated embodiment. An open end of the connector tube 22 is configured to be placed in fluid communication (e.g., syringe). It should be appreciated that the connector tube 22 may be used to introduce fluids into the body of the patient. For example, a syringe placed at the end of the connector tube 22 may be used to provide saline, or for aspiration purposes. The remote end of the connector tube 22 may be configured to directly fit into an adaptor that may, in turn, fit into a catheter, in some embodiments. Similarly, other types of attachment structures may be envisioned at the remote end of the connector tube 22 to adequately and securely attach to the catheter.

The proximal end 16 of the tubular body defines a cavity such that the proximal end 16 of the tubular body may be connected to the valve assembly 20. As shown in the illustrated embodiment, the cavity formed at the proximal end of the tubular body 12 allows for the valve assembly 20 to fit snugly into the cavity, thereby providing a means to attach to each other. The cavity is a small hollowed out cylindrical section at the tubular body, as seen in the cross-sectional view of FIG. 1. The diameter of the cavity is greater than that of the lumen 32. The cavity does not span the length of the tubular body 12, but rather is a small section at the proximal end 16.

The rotatable connector 18 may be axially connected at the distal end 14 of the tubular body 12 such that the tube 28 fits into the lumen 34 of the rotatable connector 18. The rotatable connector 18 may be connected to a catheter or port to the patient's cardiovascular or other intravenous system (not shown) on the proximal end, and the tubular body 12 on the distal end. The rotatable connector 18 is attached to the tube 28 of the tubular body 12. The rotatable connector 18 acts as an adaptor to allow the tubular body 12 to be attached to a port or catheter that is inserted into a patient's body. In other embodiments the connector 18 is permanently attached to the tubular body 12. The rotatable connector 18 allows for the tubular body 12 to rotate without rotating or torqueing the catheter inside the patient's body.

In the illustrated embodiment, the valve assembly 20 is coupled to the proximal end 16 of the tubular body 12. In particular, the valve assembly 20 comprises an inner screw body 36 and an outer screw body 38 that are configured to be screw-threaded in relation to each other. A distal end 40 of the inner screw body 36 is configured to be nestled into the proximal end of the tubular body 12, as shown in the illustrated embodiment. An outer diameter of the distal end 40 of the inner screw body 36 is configured such that it fits into the proximal end 16 of the tubular body 12. A ridge 44 may be created on the inner screw body 36, such that an outer diameter of the ridge 44 is substantially similar to the outer diameter of the proximal end 16 of the tubular body 12. The inner screw body 36 and the outer screw body 38 together define a lumen 48 that is continuous to the lumen 32 when aligned into the tubular body 12, as shown in the illustrated embodiment.

The outer screw body 38 is configured to be retreated from or advanced into the inner screw body 36. The inner screw body 36 and the outer screw body 38 are configured to advance and retreat from each other in screw thread relation, through the inner grooves 70 on the inner screw body and the complementary outer grooves 72 on the outer screw body 38.

As shown in the illustrated embodiment, the proximal end 42 of the inner screw body is configured to fit into a distal end 52 of the outer screw body. In one or more embodiments, the proximal end 54 of the outer screw body 38 may have indentations or aspects (e.g., a larger diameter, etc.) that may allow for a user to easily screw or unscrew the outer screw body from the inner screw body 36 in order to lock or unlock a catheter that is disposed within the continuous lumen of the hemostasis valve 100, as will be described in further detail below.

As shown in the illustrated embodiment, the lumen of the rotating connector 34, the lumen of the tube 28, the lumen of the tubular body 12, the lumen of the valve assembly 48 are all designed to be continuous with each other such that a catheter or guidewire may easily fit and be translated through the continuous lumen 68 of the hemostasis valve 100. The continuous lumen 68 of the hemostasis valve 100 allows for the passage of a catheter or guidewire to be passed through.

Notably, the hemostasis valve 100 comprises a combination of seals that enables a catheter running through the continuous lumen to be laterally translated while substantially maintaining aspiration and/or preventing fluid loss through the catheter, and also enabling the catheter to be locked in place as needed. To this end, a slit seal 62 is disposed in between the tubular body 12 and the outer screw body 36, and a compressible seal 64 is disposed between the inner screw body 36 and the outer screw body 38. A diameter of the slit seal 62 may be configured to be substantially equal to the inner diameter of a cavity formed at the proximal end 16 of the tubular body.

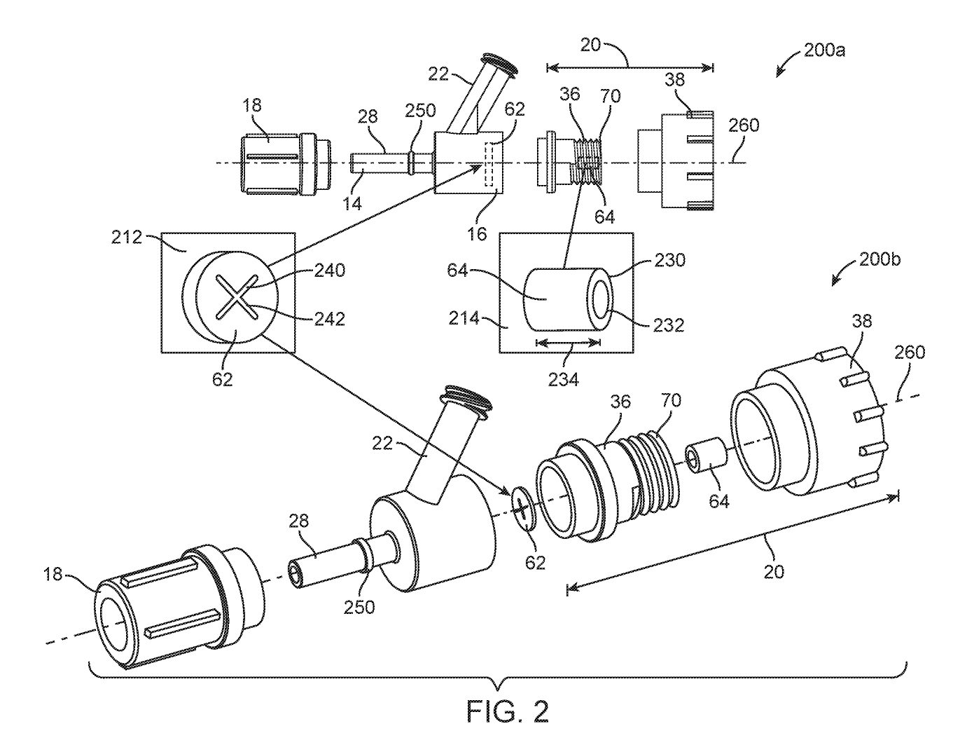

The slit seal 62 may be a cross-slit seal, as shown in FIG. 2, or may be any type of duckbill type valve. Such valves seal on themselves in the absence of an inserted instrument, such as a catheter. Such seals may be formed from an elastomeric tube formed with inward folded walls to hold an end of the tube together so as to form various number of slits, such as one (single duckbill), two (double duckbill; cross slit), three (trifold). In the absence of a catheter or guidewire running through the continuous lumen, the slit seal 62 maintains its shape such that the two crossed slits are closed, thereby sealing against leakage across the seal. When the catheter or guidewire is inserted through the slit seal 62, the seal may resiliently deform to allow the instrument to pass through, and may seal around the inserted catheter or guidewire. When the catheter or guidewire is removed, the slit seal 62 returns to its closed position. The slit seal may have any number of slits, but for the illustrative purposes, the embodiments described herein describe a cross-slit seal.

Advantageously, the slit seal 62 allows a catheter to be inserted through it when the catheter is inserted into the continuous lumen of the hemostasis valve 100. This slit seal preserves the seal (and vacuum) even when the catheter is laterally translated because the slit seal substantially closes around the inserted catheter, thereby maintaining the seal. As shown in the illustrated embodiment, the slit seal 62 is placed between the distal end 40 of the inner screw body 36 and the cavity formed at the proximal end 16 of the tubular body 12. Thus, the slit seal 62 sits snugly between the tubular body 12 and the inner screw body 36 such that it is substantially secured between the two parts of the hemostasis valve 100.

The compressible seal 64 is a resiliently deformable annular ring made of a material that deforms when radial pressure is applied to it. In one or more embodiments, the material comprising the compressible seal 64 should be sufficiently resilient to enable the compressible seal 64 to spring back to its original shape when the compressible force is removed. In one or more embodiments, the compressible seal may be made from silicon rubber. In other embodiments, the compressible seal may be made from any other elastomeric material.

In the illustrated embodiment, the compressible seal 64 is configured to be fitted into a cavity formed at the proximal end of the inner screw body 36 (i.e., the compressible seal maintains contact with the wall of the cavity), such that the lumen of the annular ring is continuous with the lumen 48 of the valve assembly 20. The compressible seal 64 has an exterior surface extending between a distal end 66 and proximal end 68. The exterior surface of the compressible seal 64 has an outer diameter that is approximately equal to the diameter of the inner surface of the inner screw body 38. In one or more embodiments, the compressible seal 64 may have a generally tubular shape, as illustrated in FIG. 2. The compressible seal 64 is configured to fit under the screw thread grooves of the inner screw body 36 such that when the grooves of the outer screw body 38 screw into the grooves of the inner screw body 36, radial pressure is exerted on the compressible seal 64. This radial pressure, in turn, serves to secure the catheter or guidewire running through the hemostasis valve, thereby locking the catheter in place.

It should be appreciated that although the illustrated embodiment depicts a rotating screw design, the compressible seal may be similarly used with any comparable valve parts that allow for compression of the seal. For example, other embodiments may utilize a button that may be pushed to deliver the radial force, or perhaps a lever that switches into a position that causes radial force. Thus, many other physical embodiments of the valve assembly may be similarly envisioned. Thus, the combination of seals, the slit seal 62 and compressible seal 64, allows for lateral translation of a catheter or guidewire running through the continuous lumen of the hemostasis valve 100 while substantially maintaining the seal, but simultaneously allows for locking of the catheter or guidewire at a desired location, as needed.

Referring now to FIG. 2, a disassembled plain view 200a and a disassembled perspective view 200b are illustrated simultaneously to depict the combination of seals as located in the hemostasis valve 100. As shown in 200a and 200b, the tube 28 of the tubular body 12 fits into the rotatable connector 18 on the distal end 14. In the illustrated embodiment, a ridge structure 250 is formed on the tube 28; this ridge structure allows the tube 28 of the tubular body 12 to be snapped into the rotatable connector 18, in one or more embodiments. Once snapped in, the rotatable connector 18 stays connected to the tubular body 12, but may be rotated along the axis 260.

The connector tube 22 extends at an angle from the tubular body 12 in the illustrated embodiment, and may be connected to device luer for flushing fluids or aspirating. The valve assembly 20, including the inner screw body 36 and the outer screw body 38, fit into the cavity formed at the proximal end 16 of the tubular body 12. The inner screw body 36 and the outer screw body 38 are configured to advance and retreat from each other in screw thread relation, through the inner grooves 70 on the inner screw body and the complementary outer grooves 72 on the outer screw body 38.

Although not illustrated in FIG. 2, the continuous lumen of the hemostasis valve has a diameter large enough such that a catheter or guidewire (not shown) passes through the lumen, from the proximal end to the distal end along the axis 2.

The illustrated embodiments 200a and 200b better illustrate the placement of the seals 62 and 64 in the hemostasis valve 100. The slit seal 62 is disposed in the cavity on the proximal end 16 of the tubular body 12, between the tubular body 12 and the inner screw body 36. In the close-up view 212 of the slit seal 62, two orthogonal slits 240 and 242 are cut into the seal material to form the cross-slit seal 62. The disassembled view 200b shows the slit seal 62 as being disposed between the tubular body 12 and the inner screw body 36.

The compressible seal 64 is disposed inside the inner screw body 36, as shown in 200a. View 200b shows the compressible seal 64 as a separate part that is nestled into a cavity formed inside the inner screw body 36. When the outer grooves (not shown) of the outer screw body 38 are screwed into the complementary grooves 70 of the inner screw body 36, radial pressure is applied to the compressible seal, which in turn clamps down on a catheter or guidewire (not shown) that runs through the continuous lumen of the hemostasis valve 100.

The close up view 214 of the compressible seal 64 shows an annular ring having an outer diameter 230 and an inner diameter 232. The inner diameter 232 defines a lumen 236 that is configured to be large enough to allow the catheter or guidewire to pass through it. However, the inner diameter 232 may be close to the diameter of the catheter or guidewire, such that when radial pressure is applied, the compressible seal locks the catheter in place. The outer diameter 230 is substantially similar to the inner diameter of the inner screw body 36 at the proximal end 42, in one or more embodiments.

The length 234 of the compressible seal 64 may be selected so that the entirety of the length fits into the cavity at the proximal end 42 of the inner screw body 36. More particularly, in one or more embodiment, the length 234 of the compressible seal 64 may be set such that the first few screw grooves (when the outer screw body 38 screws into the inner screw body 36) do not apply radial pressure on the compressible seal, thereby allowing the catheter to be “unlocked,” but only causes locking, when the outer screw body 38 moves forward a predetermined number of screw threads into the inner screw body 36. For example, the outer screw body 38 when attached to the inner screw body by screwing only the first three screw thread grooves may maintain an “unlocked” position, but when the outer screw body 38 retreats further past the fourth screw thread groove, radial pressure may then be applied to the compressible seal 64, thereby causing locking of the catheter running through the hemostasis valve 100.

When the outer screw body 38 is not retreated enough into the inner screw body 36 to apply radial pressure on the compressible seal 64, a catheter or guidewire running through the hemostasis valve 100 remains unlocked, and may be laterally translated. The slit seal 62 ensures that lateral translation does not compromise aspiration, or cause loss of fluids, because the slit seal 62 seals snugly around the catheter or guidewire. For example, the connector tube 22 may be connected to saline, or may be used for aspiration. The slit seal 62 allows for aspiration to continue because the slit seal 62 maintains vacuum within the hemostasis valve 100.

When the outer screw body 38 is retreated enough into the inner screw body 36 so as to apply radial pressure on the compressible seal 64, the catheter or guidewire is locked into position. This is advantageous, because once an optimal location for the catheter has been found, the surgical procedure may benefit from the catheter being in a fixed location rather than move around unnecessarily. Accidental movement of the catheter may lead to mistakes and/or longer procedure times.

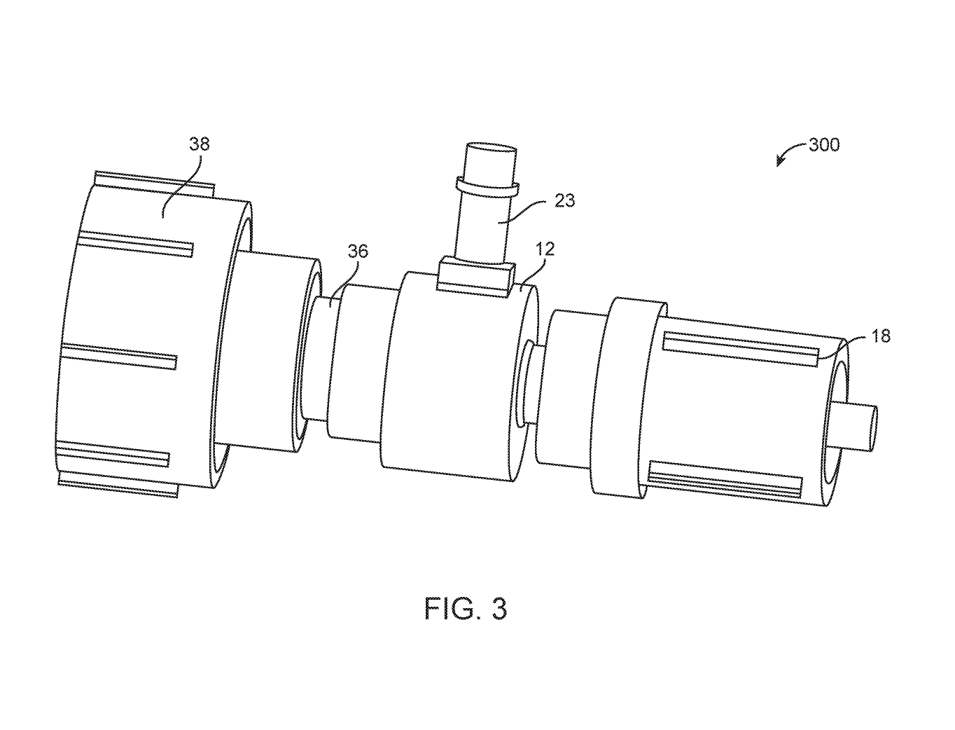

Referring now to FIG. 3, an alternate embodiment 300 of the connector tube 22 in relation to the tubular body 12 is illustrated. In contrast to the embodiments illustrated in FIGS. 1 and 2, the connector tube 23 of FIG. 3 is disposed orthogonally to the tubular body 12.

Although the current disclosure illustrates the combination of seals in a hemostasis valve, it should be appreciated that the concepts outlined herein regarding the combination of seals may be similarly applied to any valve apparatus, and should not be read as limiting. Further, it can be appreciated that the examples described above and depicted in the accompanying figures are only illustrative, and that other embodiments and examples also are encompassed within the scope of the appended claims. For example, while the flow diagrams provided in the accompanying figures are illustrative of exemplary steps; the overall image merge process may be achieved in a variety of manners using other data merge methods known in the art. The system block diagrams are similarly representative only, illustrating functional delineations that are not to be viewed as limiting requirements of the disclosed inventions. It will also be apparent to those skilled in the art that various changes and modifications may be made to the depicted and/or described embodiments (e.g., the dimensions of various parts), without departing from the scope of the disclosed inventions, which is to be defined only by the following claims and their equivalents. The specification and drawings are, accordingly, to be regarded in an illustrative rather than restrictive sense.

What is claimed is:

1. A valve apparatus, comprising:
a valve body (100) comprising a plurality of detachable parts, wherein respective housings of the detachable parts are connected together in an axial fashion to form the valve body, the valve body (100) having a proximal end (54) and a distal end (18) and defining a lumen (68) for passage of an elongate medical instrument therethrough, wherein the distal end (18) of the valve body is configured to be coupled to a port of an introducer system accessing a blood vessel of a patient;
a compressible seal (64) disposed within a screw thread portion (70) of a first detachable part (36) of the valve body, the compressible seal (64) having a lumen (236) for passage of the medical instrument, wherein the first detachable part (36) of the valve body is threadedly connected to a second detachable part (38) of the valve body such that relative rotation of the first (36) and second detachable parts (38) causes them to advance or retract relative to each other, and wherein the compressible seal (64) is configured to be compressed inside the first detachable part (36) when the second detachable part (38) is screwed into the first detachable part (36) to thereby secure the medical instrument in place; and
a slit seal (62) disposed between a third detachable part (12) and the first detachable part (36), the slit seal (62) disposed such that the medical instrument passes through the slit seal (62), maintaining a seal as the medical instrument undergoes lateral translation through the lumen (68) of the valve body (100).

2. The valve apparatus of claim 1, wherein the slit seal (62) is cross-slit.

3. The valve apparatus of claim 1, wherein an outer diameter (230) of the compressible seal (64) is substantially equal to an inner diameter of a cavity formed at a proximal end (42) of the first detachable part (36).

4. The valve apparatus of claim 1, wherein a diameter of the slit seal (62) is substantially equal to an inner diameter of a cavity formed at a proximal end (16) of the third detachable part (12).

5. The valve apparatus of claim 1, wherein an inner diameter of the compressible seal (64) is substantially equal to an outer diameter of the medical instrument.

6. The valve apparatus of claim 1, wherein the compressible seal (64) is made from a rubber material.

7. The valve apparatus of claim 1, wherein the slit seal (62) is made from a silicone material.

8. The valve apparatus of claim 1, wherein the compressible seal (64) is proximal in relation to the slit seal (62).

9. The valve apparatus of claim 1, wherein the valve apparatus is a hemostasis valve.

10. The calve apparatus of claim 1, wherein the medical apparatus is one of a catheter and a guidewire.

11. A valve apparatus comprising a plurality of detachable parts, wherein respective housings of the detachable parts are connected together in an axial fashion to form a valve body (100), the valve apparatus comprising:
a continuous lumen (68) for passage of a catheter or guidewire through the valve body (100);
a compressible seal (64) positioned within the lumen (68) in between a first detachable part (36) and second detachable part (38) of the valve body (100), the compressible seal (64) having a first lumen (236) continuous with the continuous lumen (68), whereby, when the catheter or guidewire is present in the lumen, a movement of the first detachable part (36) relative to the second detachable part (38) causes a radial compression force to be exerted on the compressive seal (64), thereby locking the catheter or guidewire in place; and
a slit seal (62) having a second lumen continuous with the continuous lumen (68), the slit seal (62) maintaining a seal even when the catheter or guidewire undergoes lateral translation through the lumen (68).

12. The valve apparatus of claim 11, wherein the first detachable part (36) and the second detachable part (38) comprise complementary screw thread grooves (70,72), and wherein the relative movement comprises screwing the first detachable part (36) in relation to the second detachable part (38).

13. The valve apparatus of claim 11, wherein the slit seal (62) is cross-slit.

14. The valve apparatus of claim 11, wherein an outer diameter (230) of the compressible seal (64) is substantially equal to an inner diameter of a cavity formed at a proximal end (42) of the first detachable part (36).

15. The valve apparatus of claim 11, wherein the compressible seal (64) is made from a rubber or silicon material.

16. The valve apparatus of claim 11, wherein the compressible seal (64) is proximal in relation to the slit seal (62).

17. The valve apparatus of claim 11, wherein the valve apparatus is a hemostasis valve.

18. A hemostasis valve apparatus, comprising:
a valve body (100) comprising a plurality of detachable parts, wherein respective housings of the detachable parts are connected together in an axial fashion to form the valve body, the valve body (100) having a proximal end (54) and a distal end (18) and defining a lumen (68) for passage of an elongate medical instrument therethrough, wherein the distal end (18) of the valve body is configured to be coupled to a port of an introducer system accessing a blood vessel of a patient;
a compressible seal (64) disposed within a screw thread portion (70) of a first detachable part (36) of the valve body, the compressible seal (64) having a lumen (236) for passage of the medical instrument, wherein the first detachable part (36) of the valve body is threadedly connected to a second detachable part (38) of the valve body such that relative rotation of the first (36) and second detachable parts (38) causes them to advance or retract relative to each other, and wherein the compressible seal (64) is configured to be compressed inside the first detachable part (36) when the second detachable part (38) is screwed into the first detachable part (36) to thereby secure the medical instrument in place; and
a cross-slit seal (62) disposed between a third detachable part (12) and the first detachable part (36), wherein the cross-slit seal (62) disposed such that the medical instrument passes through the cross-slit seal (62), maintaining a seal as the medical instrument undergoes lateral translation through the lumen (68) of the valve body (100),
wherein an outer diameter (230) of the compressible seal (64) is substantially equal to an inner diameter of a cavity formed at a proximal end (42) of the first detachable part (36), and wherein a diameter of the slit seal (62) is substantially equal to an inner diameter of a cavity formed at a proximal end (16) of the third detachable part (12).

19. The hemostasis valve apparatus of claim 18, wherein an inner diameter of the compressible seal (64) is substantially equal to an outer diameter of the medical instrument, and wherein the compressible seal (64) is proximal in relation to the slit seal (62).

20. The hemostasis valve apparatus of claim 18, wherein the medical apparatus is one of a catheter and a guidewire.
